(19) United States
(12) Patent Application Publication
(10) Pub. No.: US 2026/0034377 A1
(43) Pub. Date: Feb. 5, 2026

(54) DEVICE FOR CARDIOLOGIC MAGNETIC AND OPTICAL STIMULATION

(71) Applicant: Imutec SAS, Paris (FR)

(72) Inventor: Nadia Volf, Paris (FR)

(21) Appl. No.: 19/357,469

(22) Filed: Oct. 14, 2025

Related U.S. Application Data
(63) Continuation-in-part of application No. 19/305,900, filed on Aug. 21, 2025.
Application No. 19/305,900 is a continuation of application No. 17/695,089, filed on Mar. 15, 2022, now Pat. No. 12,415,084.

(51) Int. Cl. A61N 2/00; A61N 2/02; A61N 5/06

(52) U.S. Cl. CPC A61N 2/002; A61N 2/004; A61N 2/02; A61N 5/0613; A61N 2005/0652; A61N 2005/0659; A61N 2005/0662

(57) ABSTRACT

A portable, wearable system and method for real-time determination and modification of a patient's cardiac electromagnetic activity. The device has a sensor array with ECG electrodes, a controller executing software, a stimulator array with at least one electromagnet and one light-emitting diode, and a power supply, optionally integrated into a belt or chest harness. The system continuously acquires ECG signals, calculates a force momentum Bion (β) based on cardiac cycle parameters, and generates synchronized electromagnetic and optical stimulation in real time, modulating output according to the patient's heart activity. Safety features include detection of electrode contact, noise, and arrhythmia, with automatic blocking of stimulation and alert generation. The device further provides wireless communication for data transmission and remote monitoring, and supports amplitude- and frequency-modulated stimulation profiles. The method includes real-time signal processing, adaptive stimulation, and configurable alerts for cardiac anomalies, enabling noninvasive cardiac monitoring and intervention.

### CROSS REFERENCE TO RELATED APPLICATIONS

This application is a continuation in part of U.S. patent application Ser. No. 19/305,900 filed Aug. 21, 2025, which is a continuation of U.S. patent application Ser. No. 17/695,089 filed Mar. 15, 2022, now U.S. Pat. No. 12,415,084 dated Sep. 16, 2025.

### FIELD OF INVENTION

The present invention generally relates to the real-time sensing and measurement of the heart's electromagnetic field, synchronically generating and imposing an electromagnetic and/or optical field on the patient's body and heart area. This process, based on real-time measured cardiac electromagnetic activity, aims to influence the efficiency of biological processes and organ function. More particularly, the invention relates to a portable, wearable device, such as a belt or harness, that acquires ECG signals, processes heart activity data, and generates synchronized magnetic and optical stimulations in phase with the heart's electrical activity. The device further includes wireless communication capabilities, configurable stimulation profiles, enhanced safety features, and a novel assembly method to minimize mechanical stress and electrical interference.

### BRIEF DESCRIPTION OF THE DRAWINGS

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the inventive concepts disclosed herein.

The accompanying drawings, which are incorporated in and constitute a part of the specification, illustrate embodiments of the disclosure and together with the general description of the disclosure given above and the detailed description of the drawings, given below, explain the principles of the disclosure.

In the drawings:

FIG. 1 is a schematic of the Electromagnetic Stimulation Device, in an embodiment.

FIG. 2 is a method flow chart of the invention, in an embodiment.

FIG. 3 is a graph of an ECG trace.

FIG. 4A shows an example of the device configured as a belt.

FIG. 4B shows an example of the device configured as a chest harness.

FIG. 4C is a schematic top view of an embodiment of the device.

FIG. 4D is a schematic bottom view of an embodiment of the device.

FIG. 5 is a schematic of one embodiment of a controller of the device, configured as a microcontroller.

FIG. 6 is a method flow chart of the invention, in an embodiment.

FIG. 7 shows an embodiment of a stimulation profile where the frequency of the stimulation depends on the phases encountered.

FIG. 8 is an exemplary field diagram.

The drawings are not necessarily to scale. In certain instances, details that are not necessary for an understanding of the disclosure or that render other details difficult to perceive may have been omitted. It should be understood, of course, that the disclosure is not necessarily limited to the embodiments illustrated herein.

### DETAILED DESCRIPTION

The present invention provides its benefits across a broad spectrum of endeavors. It is applicant's intent that this specification and the claims appended hereto be accorded a breadth in keeping with the scope and spirit of the invention being disclosed despite what might appear to be limiting language imposed by the requirements of referring to the specific examples disclosed. Thus, to acquaint persons skilled in the pertinent arts most closely related to the present invention, a preferred embodiment of the system is disclosed for the purpose of illustrating the nature of the invention. The exemplary method of installing, assembling and operating the system is described in detail according to the preferred embodiment, without attempting to describe all the various forms and modifications in which the invention might be embodied. As such, the embodiments described herein are illustrative, and as will become apparent to those skilled in the art, can be modified in numerous ways within the scope and spirit of the invention, the invention being measured by the appended claims and not by the details of the specification.

Although the following text sets forth a detailed description of numerous different embodiments, the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined herein, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, subparagraph (f).

### Electromagnetic Stimulation Device Sensing, Measurement, and Output System

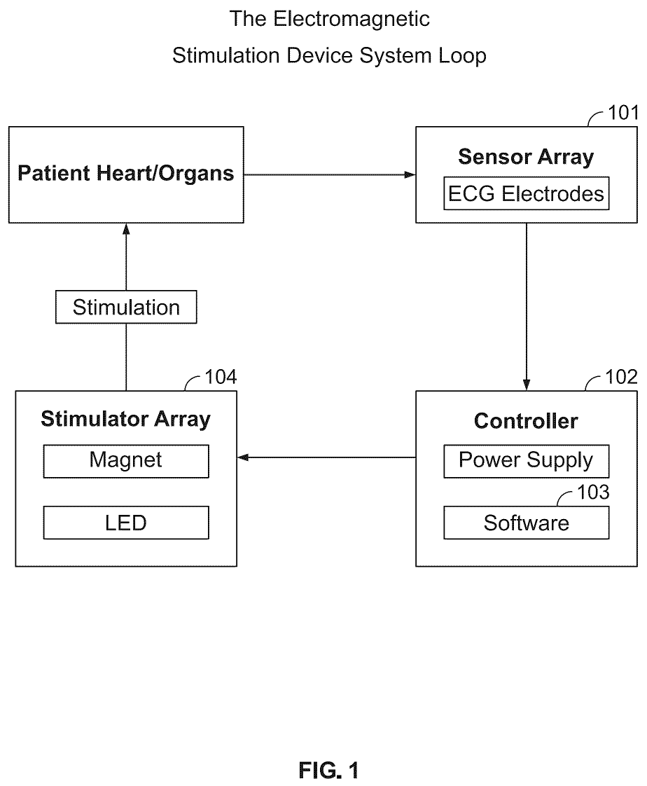

With reference to FIG. 1, the present invention contemplates an electromagnetic sensing, measuring, and output system. In some embodiments, the sensing, measuring, and output system is configured to non-invasively attach to a patient and comprises a sensor array 101, a controller 102, software 103, a stimulator array 104, and a power supply. In some embodiments, the sensor array 101 includes one or more sensors, including, for example, ECG electrodes.

In some embodiments, the sensor array 101 is configured to detect the electromagnetic activity of the patient's heart and send signals to the controller 102 via a data communication link. The controller 102 is configured to execute software 103, receive signals from the sensor array 101, and translate the signals into digital readable data. The software 103 is configured to receive data from the controller 102, analyze that data, present the data visually to the user in human-readable format, and send the data to the stimulator array 104 via a data communication link. In some embodiments, the stimulator array 104 includes at least one or more electromagnets. In some embodiments, the stimulator array 104 includes at least one or more light-emitting diodes. In some embodiments, the light-emitting diodes can emit light between visible and infrared light. In some embodiments, the electromagnets and light-emitting diodes of the stimulator array 104 emit an electromagnetic field (comprising, in some embodiments, light stimulation) that affects the electromagnetic activity of the patient's heart.

As noted, the sensor array 101 can comprise one or more sensors that are configured to detect the electromagnetic activity of a patient's heart. A controller 102 transfers the sensor array data into readable data and processes that readable data using software 103. The software program 103 is used to visually present data to the user and, in some embodiments, send data to the stimulator array 104 via a data communication link. The stimulator array 104 is used to create an electromagnetic field (including, in some embodiments, light stimulation) to modify the electromagnetic activity of the patient's heart. A data communication link can include, for example, ethernet, USB, PCI, Bluetooth, or wireless.

The invention also contemplates a portable version of the device in which the sensor array 101, controller 102, software 103, stimulator array 104, and the power supply all in an integrated housing. In some embodiments, the portable version of the invention is the size of a fist. A power supply can include, for example, an AC adaptor, a mains adaptor, a battery, or a rechargeable battery.

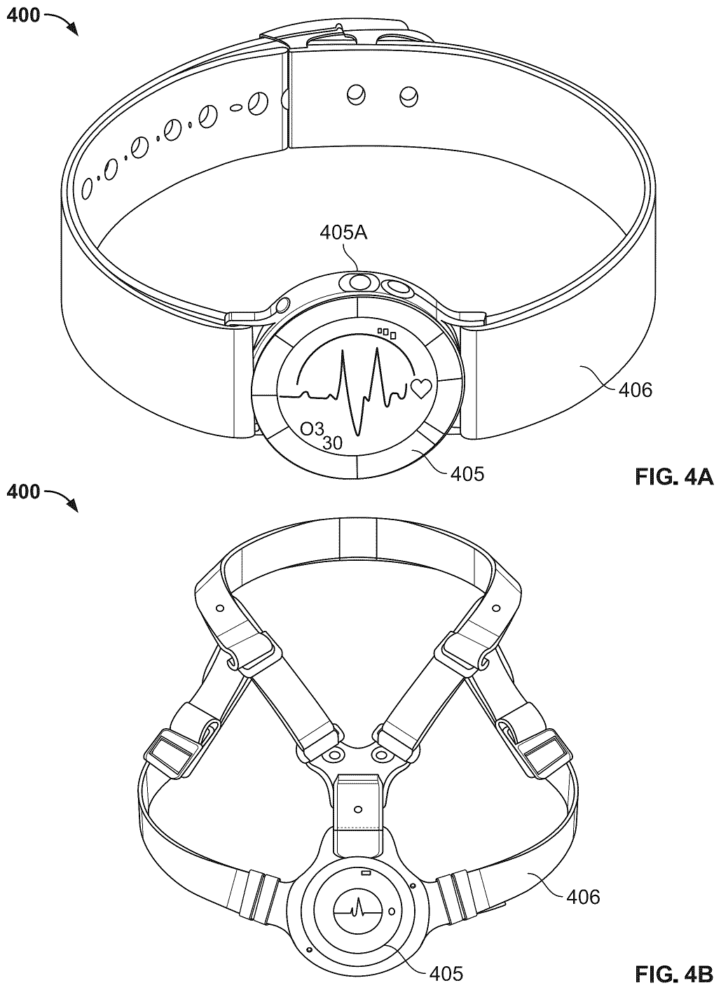

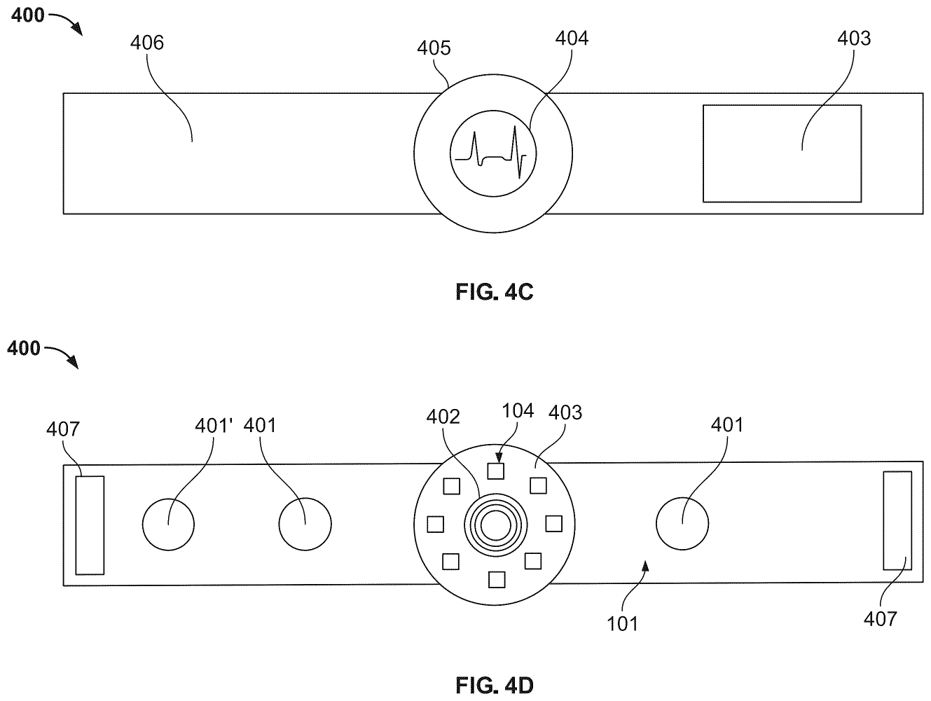

With reference to FIGS. 4A through 4C, shown is a portable version of the device 400 implemented as a portable belt (FIG. 4A) or harness (FIG. 4B) designed to be worn on the chest of a user, close to the heart, including a sensor array 101 comprising at least two ECG electrodes 401 positioned on each side of the heart. An optional third electrode 401′ may be included for improved signal integrity and common noise filtering. The electrodes may be “dry” or “wet” and are configured to maintain constant contact with the user's skin during operation. In some embodiments, the stimulator array 104 comprises an electromagnet 402 and one or more light-emitting diodes 403. In some embodiments, the electrodes may also be integrated into a wearable shirt, with conductive fabric woven into the shirt and connected to the device via mechanical clips or wires. In some embodiments, the device 400 further includes a portable, optionally rechargeable, battery 403 powering the entirety of the system; an embedded or optional LCD screen 404 for real-time visual reproduction of the acquired ECG signal, configuration parameters, and indicators such as battery charge, temperature, and stimulation efficiency; an enclosure 405 including one or more navigation buttons (405′, see FIG. 4A) for device operation, stimulation length and intensity configuration, and alert management, which may be replaced or supplemented by a wireless application interface; an adjustable belt 406 with adjustable fasteners 407. A wireless charging coil may be integrated into the belt or harness for contactless battery recharging, in addition to wired charging options (e.g., USB or power plug).

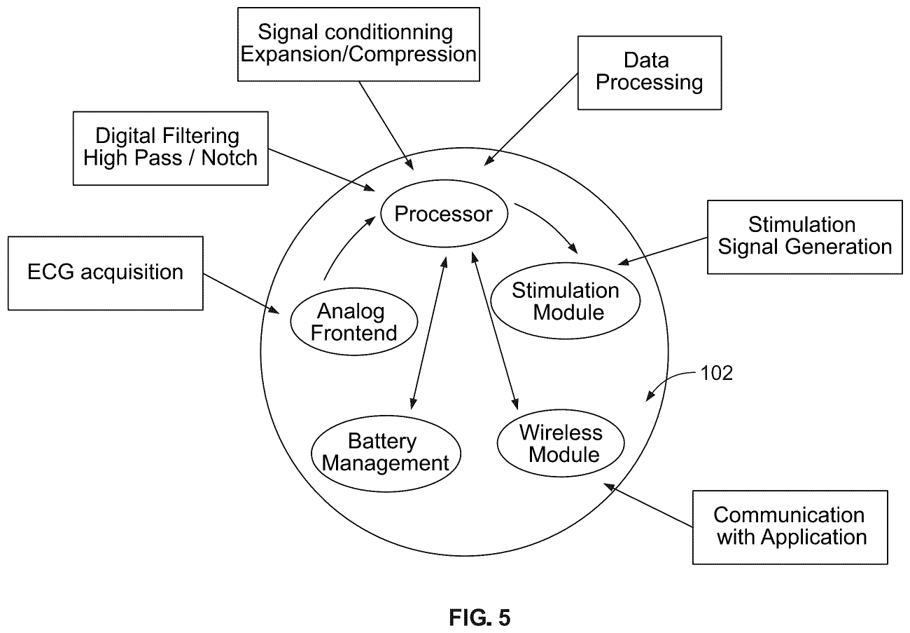

With reference to the system diagram shown in FIG. 5, retained in the enclosure 405 is a controller 102 configured as a data processing unit for filtering, decision making, and configuring second electronic devices that generate a calculated dynamic magnetic field and optical emission as described here. In some embodiments, the controller 102 comprises a microcontroller 500 having a processor, an analog frontend, a battery management module, and a stimulation module interconnected to the stimulation array 104. In some embodiments, the microcontroller includes or is interconnected with a wireless communication module enabling data transfer to external interfaces such as a smartphone application or computer GUI and allowing remote configuration of the device. The microcontroller is configured, in some embodiments, to handle ECG acquisition, digital filtering, signal conditioning, data processing, and stimulation signal generation.

In some embodiments, the navigation buttons 405′ are for operation of: turning on or off the device, configuring the maximum length of the stimulation, configuring the intensity of the magnetic field and the optical emission, observing the time remaining in the currently configured stimulation cycle, observing the state of the integrated or removable battery (charge and temperature), observing the efficiency of the stimulation: as noted in the patent defining the “BION” (described below), observing the ECG signal in real time, configuring visual and sound alerts that include tachycardia, arrhythmia and any detectable anomaly that can be extracted by an ECG acquisition and state of the art algorithms, and transmitting the data to an accredited destination by connecting to an application of a smartphone or wirelessly to a basestation that has access to Internet. In some embodiments, control of the device 400 can be handled, in whole or in part, by a mobile application.

### Electromagnetic Stimulation Device Sensing, Measurement, and Output Method

In some embodiments, the sensor array 101 continuously and in real-time monitors and captures the electromagnetic activity data of the patient's heart via at least one or more ECG electrodes. The sensor array 101 sends the electromagnetic activity data to a controller 102 and then the controller 102 feeds the data to software 103 that analyzes and records the measured electromagnetic data. This allows the user to analyze and detect electromagnetic activity of the patient's heart.

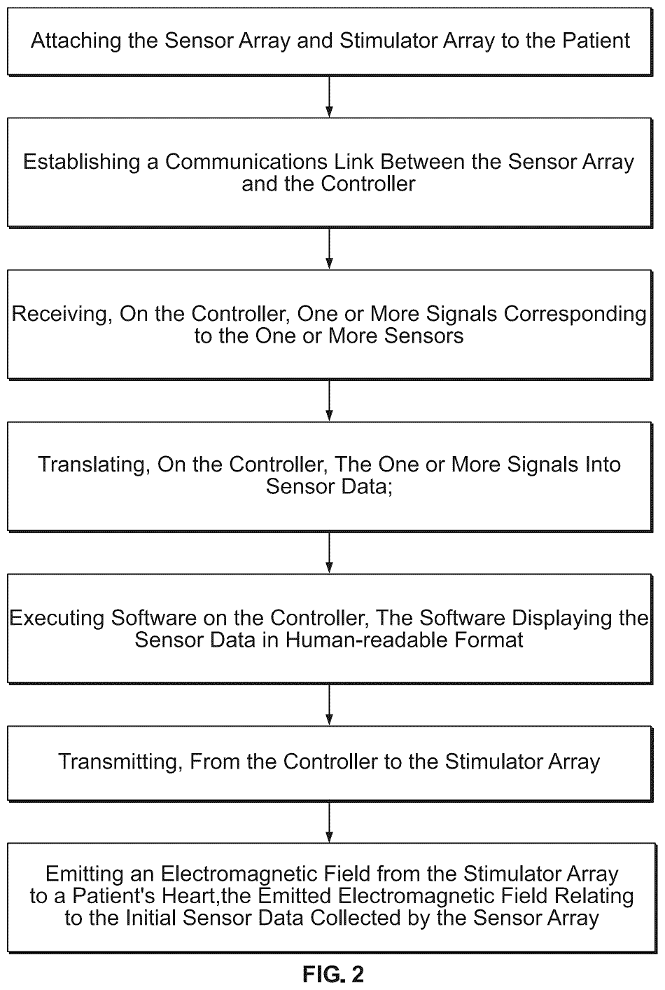

With reference to FIG. 2, once the device is activated, the sensor array 101 begins to measure the electromagnetic activity of the patient's heart. The electromagnetic activity measured by the sensor array 101 are sent to the controller 102 executing software 103 via a data communication link. The controller 102 converts the electromagnetic activity detected by the sensor array 101 into a digital form that can be read by software 103. The software 103 analyzes and displays the electromagnetic activity in a graph type report to the user. Based on the sensor data received from the controller 102, the stimulator array 104 emits an electromagnetic field that affects the electromagnetic activity of the patient's heart. In some embodiments, based on the graph form electromagnetic activity data the user is able to determine through observation how to proceed with the recharging process and/or whether the recharging process is complete.

In some embodiments, the data in the software 103 can be post-processed and the program can incorporate a feedback loop back to the stimulator array 104 to correct, modify, or enhance the electromagnetic activity of the patient's heart.

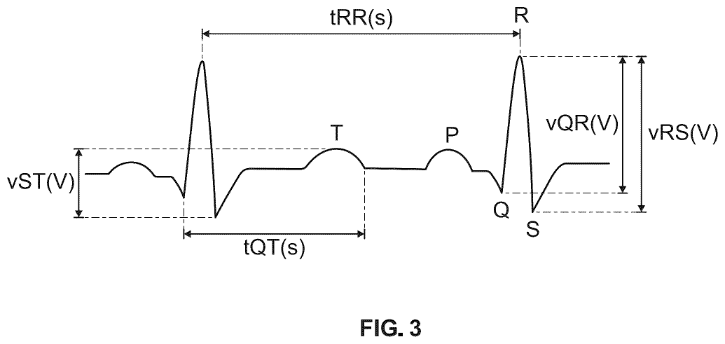

The controller 102 automatically and in real-time determines the value of the internal electrophysiological state of the heart and by extension the electromagnetic fields that are naturally created and transmitted throughout the patient, or the novel value Bion (β). Bion (β) represents the force momentum which is a measure of the average efficiency of all biochemical processes that take place in the heart. Bion (β), or force momentum, is calculated by dividing the summation of the amplitudes of QR+RS and ST waves (measured in mV), which represents the total global potential action, by the corrected time, tQTc. The corrected time tQTc avoids the influence of the variations of the heart rate modulated by breath, medications or pathological conditions. The equation defining this relationship may thus be expressed as shown below, where vQR is the charge potential, vRS is the discharge potential, and vST is the recharge potential and the corrected time tQTc is calculated by dividing the time tQT by the square root of the interval tRR, wherein tRR is the duration of an entire ECG cycle (identical points in an ECG recording). FIG. 3 is an ECG trace exemplifying the origin of these values, with the value β calculated as follows:

$$Force Momentum = \frac{vQR + vRS + vST}{\frac{tQT}{\sqrt{tRR}}}$$

The value of force momentum β in Bion dictates the electromagnetic field produced by the stimulator array 104. In some embodiments, the Bion (β) is a value describing the properties of heart contractions: the numerator measures the energy of all phases in a cardiac muscle contraction since the ECG signal reflects the intensity of the contraction. A variation in the ECG signal may also be due to better electrode placement or skin properties, but since the calculation compares the evolution of the Bion (β) throughout time on the same patient, it can be assumed that the physiological properties of the wearer are constant. In some embodiments, the Bion (β) can be considered an indicator of the average polarisation and depolarisation of heart muscle cells that occur in a cardiac cycle. Thus, the Bion (β) can be defined as an average and relative measure in time of the efficiencies of heart functions. It reflects an evolution throughout time of the capacity of the heart to perform its major function (pumping blood out of the heart). The denominator normalizes the indicator to one average heart beat such that any evolution in the cardiac rhythm is absorbed.

While the Bion (β) indicator calculates an absolute value (in V/s), the meaning is much more relevant if it is presented to a user in intervals (graduations). If the initial value of the first minute of the recording of the Bion (β) is “1”, then any increase in its value may imply the value “2”. The graduation is relative to the initial data point and describes an “Energy Potential” that evolves and betters heart functions. For example, the graduation are defined based on the following table:

| Heart State | Calculated Bion (β) Value |
|---|---|
| Healthy | 5.2 ± 1.7 |
| Heart Failure with Preserved | 3.0 ± 0.8 |
| Ejection Fraction | |
| Ejection Fraction <45 | 2.5 ± 0.7 |
| Heart Failure | 2.7 ± 0.8 |

The graduations may be considered to span from 1 to 9, even if they are not limited by the equation (the only limit is the physiology of the heart which renders improbable any higher Bion (β) value). Bion (β) is a quantitative indicator of the electromotive force of the heart—i.e. electric potential that the heart is capable of creating during the Q-T phase for each person at the time of measurement. Accordingly, in some embodiments the values of Bion (β) can be interpreted as follows:

| Bion (β) | Heart Efficiency and Energy (“Charge”) |
|---|---|
| 1 to 3 | Low Level |
| 4 to 6 | Acceptable Level |
| 7 to 9 | High Level |
| >10 | Excellent Level |

In some embodiments, the sensor array 101 includes a feedback mechanism through the ECG electrodes to detect whether the ECG electrodes are in contact with the user. If the electrodes are not in contact, the device automatically enters a low-power sleep mode to reduce power consumption, and all calculations and stimulations are paused until contact is re-established. In some embodiments, a warning graphic and/or alert sound is generated on the LCD screen if the electrodes are disconnected.

In some embodiments, controller 102 determines the baseline that represents the phase of the heart when no cardiac contraction occurs. While this baseline is present, no stimulation is emitted. The stimulation intensity is at its highest when the ECG signal from the sensor array 101 is at its highest. Any intermediary value of the ECG is linearly correlated with the stimulation intensity. In this manner, the stimulation reflects the heart's electric activity which in turn represents the intensity of the cardiac muscle contraction. The stimulation is therefore in phase with the heart's activity or is “biologically resonant.”

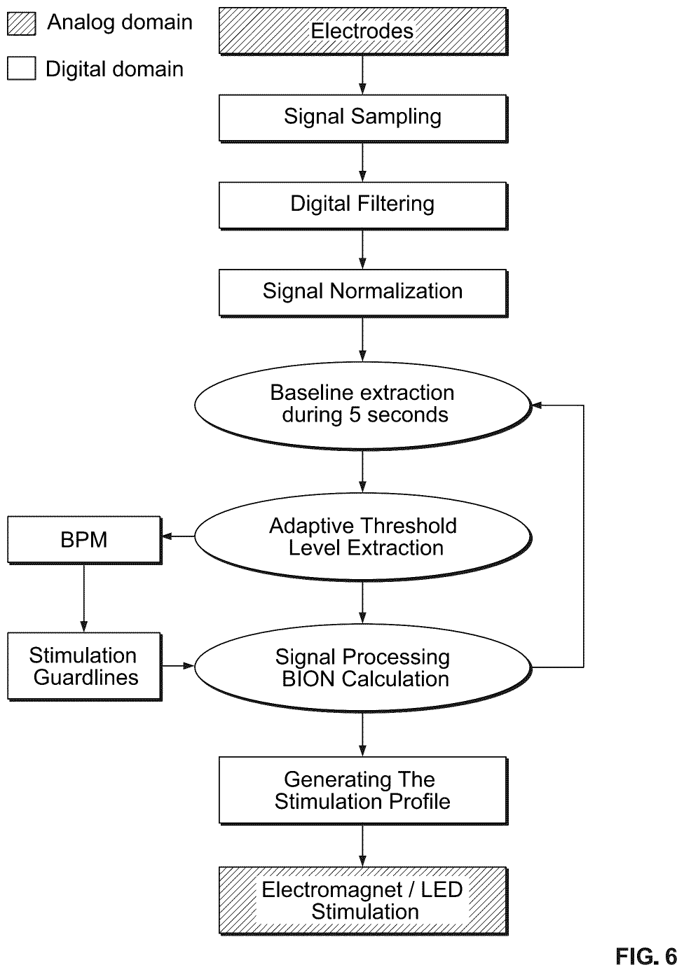

With reference to FIG. 6, in some embodiments ECG signal acquired from the sensor array 101 is processed by a digital signal processor (DSP) of the controller 102 that samples the differential signal from the electrodes at a rate of at least 200 samples per second using an instrumentation amplifier and a 12-bit analog-to-digital converter. In some embodiments, the sample rate is at least 100 samples per second with 10-bit resolution. Digital filtering is applied to remove 50/60 Hz mains noise and low-frequency drifts (<0.5 Hz). The baseline of the ECG signal is extracted by identifying the period with the lowest variability over at least 5 seconds.

In some embodiments, a threshold algorithm executing on the controller 102 blocks any stimulation if noise dominates the ECG, if the electrodes are not connected, or if arrhythmia is detected. If the ECG electrodes are not connected (with the skin of the patient) a warning graphic appears on the LCD screen as well as an alert sound. This ensures that all calculations and stimulations stop and are paused until the user reconnects the device to himself. This detection procedure takes the form of a signal that is injected from one electrode and propagates to the other through the skin: the small oscillating signal that is injected is then sampled on the receiving ECG electrode. The frequency that is injected is identical to the one that is sampled, reduced in amplitude by loses due to the resistance of the skin. If all signal constraints are met, the Bion (β) is calculated based on stored sequences of at least 10 seconds, then averaged over a minute of total acquisition.

In some embodiments, the controller 102 includes digital converters and power transistors that are regulated such that the current flowing through the stimulator array 104, namely the electromagnet and/or the light-emitting diodes, do not allow significant step variations. The stimulation profile is therefore similar to the original ECG signal.

In some embodiments, a basic stimulation profile is a copy of the acquired ECG signal: the magnetic and optical fields generated by the stimulation array 104 are identical (in variations compared to the ECG signal) to the electric field intensity created by heart contractions, with the lowest and highest intensity of the two fields corresponding to the lowest and highest value of the ECG signal. The lowest magnetic intensity is 0 Tesla and the highest 2 mT or any other value configured by the user. In some embodiments, these stimulations may be qualified as “amplitude modulated” stimulations.

An extension of this profile is a stimulation with frequency modulated magnetic and optical fields. The waves thus emitted by the device reflect different phases in the heart's contraction cycles. These phases are also tightly coupled to underlying biochemical processes that are necessary for muscle functions: a muscle contraction requires ionic transport through membranes and therefore creates a varying electric potential on both sides of the membrane that may be observed by ECG sensors. The stronger the heart contraction, the stronger the biochemical processes (which imply a higher frequency of ion transfer through cell membranes). Therefore, every phase represents the internal state of the heart, and, as an extension, the electric and magnetic fields that are naturally created and diffused throughout the patient. The stimulation frequency created is higher for more intense heart contractions than for other phases and for the baseline (when no heart activity is measured by ECG). While on the baseline, the stimulation frequency may be reduced to zero with an amplitude of zero. Higher stimulation frequencies imply higher energy transferred to the patient. This is compatible with the fact that intense heart contractions require high energy.

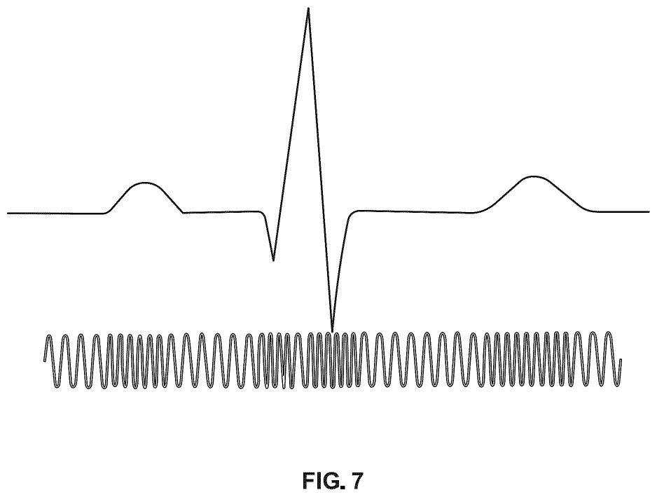

FIG. 7 depicts an alternative stimulation profile where the frequency of the stimulation depends on the phases encountered. They may be dynamically configured and can range from 0 Hz to multiples of MHz. The amplitude can also be configured but must comply with regulations (2 mT for a static magnetic exposure and higher values for other frequencies).

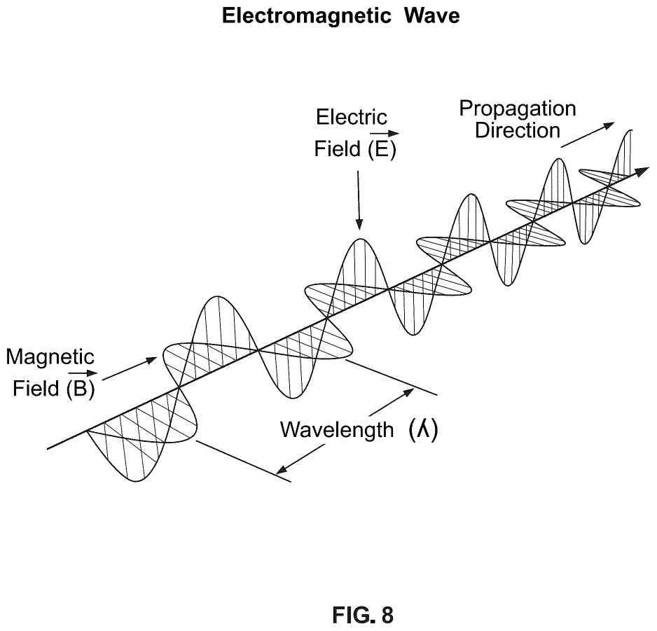

With reference to FIG. 8, because magnetic fields have polarity, there are two possibilities for the generated field: the lowest value of the magnetic field is 0 T (no field), the highest is at most the highest value for the frequency of the stimulation which is limited by regulations. The field is the same polarity (north or south) during any phase of the stimulation. In some embodiments, the magnetic field alternates between north and south while limiting the absolute highest value of the field to regulations thus effectively creating a swing of magnetic fields which is double the swing of the intensity of a single-ended polarity stimulation. The value “0” (the baseline where no magnetic field is emitted) is the point where the polarity is changed. While in the frequency modulation mode (fixed polarity or alternating polarity), the alternating magnetic field induces also an electrical field. Both fields propagate through space and the combined action is an increased interaction with biological tissues. Optical fields, on the contrary, have no polarity such that the smallest intensity is no field and the highest intensity is defined by the properties of the light-emitting diode.

In some embodiments, the controller 102 supports storage of the acquired ECG signal on a connected smart device (such as a smartphone running the aforementioned mobile application), secure transmission to a practitioner or remote IT platform, and the use of machine learning or AI algorithms to detect and alert the user to cardiac anomalies. Alerts may be visual or auditory and can be configured for conditions such as tachycardia, arrhythmia, or other detectable anomalies.

### Description of Computing Environment

Controller 102 can be connected to any external computing device smart phone, tablet computer, laptop computer, or other computing or mobile device capable of reading, and/or recording data about systems, devices, locations, and/or equipment, etc. Controller 102 can be connected to any external computing device, including any server computer, desktop computer, laptop computer, or other device capable of storing and managing data communication by and between one or more sensors of the sensor array 101 and the stimulation array 104.

In some embodiments, the controller 102 includes processing system, storage system, software, communication interface, and user interface. Processing system loads and executes software, including software 103, from storage system, including software module. When executed by controller 102, software module directs processing system to receive data, images, devices, locations, and/or equipment, etc. Such data could include any of the information described above, including but not limited to the functionality described herein. Additionally, controller 102 includes communication interface that can be further configured to transmit data to and receive data from controller 102.

The controller 102 includes a processing system that can comprise a microprocessor and other circuitry that retrieves and executes software from storage system. Processing system can be implemented within a single processing device but can also be distributed across multiple processing devices or sub-systems that cooperate in executing program instructions. Examples of processing system include general purpose central processing units, application specific processors, and logic devices, as well as any other type of processing device, combinations of processing devices, or variations thereof. Storage system can comprise any storage media readable by processing system, and capable of storing software. Storage system can include volatile and nonvolatile, removable and non-removable media implemented in any method or technology for storage of information, such as computer readable instructions, data structures, program modules, or other data. Storage system can be implemented as a single storage device but may also be implemented across multiple storage devices or sub-systems. Storage system can comprise additional elements capable of communicating with processing system.

An application interface can include data input and image display. In one example, data input can be used to collect information and data inputs from the user. It should be understood that although controller 102 is shown as one system, the system can comprise one or more systems to collect data.

Controller 102 includes processing system, storage system, software, and communication interface. Processing system loads and executes software from storage system, including software module 103. When executed by controller 102, software module 103 directs processing system to store and manage the data.

The processing system can comprise a microprocessor and other circuitry that retrieves and executes software from storage system. Processing system can be implemented within a single processing device but can also be distributed across multiple processing devices or sub-systems that cooperate in executing program instructions. Examples of processing system include general purpose central processing units, application specific processors, and logic devices, as well as any other type of processing device, combinations of processing devices, or variations thereof.

Storage system can comprise any storage media readable by processing system, and capable of storing software and data from the computing device. Data from computing device may be stored in a word, excel, or any other form of digital file. Storage system can include volatile and nonvolatile, removable and non-removable media implemented in any method or technology for storage of information, such as computer readable instructions, data structures, program modules, or other data. Storage system can be implemented as a single storage device but may also be implemented across multiple storage devices or sub-systems. Storage system can comprise additional elements, such as a controller, capable of communicating with processing system.

Examples of storage media include random access memory, read only memory, magnetic disks, optical disks, flash memory, virtual memory, and non-virtual memory, magnetic cassettes, magnetic tape, magnetic disk storage or other magnetic storage devices, or any other medium which can be used to store the desired information and that may be accessed by an instruction execution system, as well as any combination or variation thereof, or any other type of storage media. In some implementations, the storage media can be a non-transitory storage media. In some implementations, at least a portion of the storage media may be transitory. In no case is the storage media a propagated signal.

In some examples, controller 102 can include a user interface. The user interface can include a mouse, a keyboard, a voice input device, a touch input device for receiving a gesture from a user, a motion input device for detecting non-touch gestures and other motions by a user, and other comparable input devices and associated processing elements capable of receiving user input from a user. Output devices such as a graphical display, speakers, printer, haptic devices, and other types of output devices may also be included in the user interface. The user input and output devices are well known in the art and need not be discussed at length here.

The included descriptions and figures depict specific implementations to teach those skilled in the art how to make and use the best mode. For the purpose of teaching inventive principles, some conventional aspects have been simplified or omitted. Those skilled in the art will appreciate variations from these implementations that fall within the scope of the invention. Those skilled in the art will also appreciate that the features described above can be combined in various ways to form multiple implementations. As a result, the invention is not limited to the specific implementations described above, but only by the claims and their equivalents.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., the use of a certain component described above alone or in conjunction with other components may comprise a system, while in other aspects the system may be the combination of all of the components described herein, and in different order than that employed for the purpose of communicating the novel aspects of the present disclosure. Other variations and modifications may be within the skill and knowledge of those in the art, after understanding the present disclosure. This method of disclosure is intended to obtain rights which include alternative embodiments to the extent permitted, including alternate, interchangeable and/or equivalent structures, functions, ranges or steps to those claimed, whether such alternate, interchangeable and/or equivalent structures, functions, ranges or steps are disclosed herein, and without intending to publicly dedicate any patentable subject matter.

What is claimed is:

1. A portable system of determining and modifying the electromagnetic activity of a patient's heart, comprising:
a sensor array, a controller, software, a stimulator array, and a power supply retained in a portable enclosure configured for wear on the patient;
the sensor array comprising one or more ECG electrodes configured to detect the electromagnetic activity of the patient's heart in real time;
the sensor array in communication with the controller, the controller configured to translate signals received from the one or more ECG electrodes into sensor data;
the controller executing software, the software configured to output the sensor data received from the controller to enable a user to read the electromagnetic activity of the patient's heart;
the controller configured to calculate a force momentum of the heart based on the sensor data; the stimulator array comprising at least one electromagnet and at least one light-emitting diode, wherein the stimulator array is configured to emit both electromagnetic and optical signals in real-time response to the force momentum; and
wherein the electromagnetic and optical signals are modified in real time based on the electromagnetic activity of the heart.

2. The system of claim 1, wherein the portable enclosure is integrated into a wearable belt, chest harness, or shirt configured to position the one or more ECG electrodes on opposing sides of the heart and to retain the stimulator array proximate the chest.

3. The system of claim 1, wherein the power supply comprises a rechargeable battery and a wireless charging coil integrated into the belt or harness, and further comprising a wired charging interface.

4. The system of claim 1, further comprising an LCD screen coupled to the controller and one or more navigation buttons on the enclosure, the LCD screen configured to display a real-time ECG signal, configuration parameters, battery state, temperature, and stimulation efficiency.

5. The system of claim 1, wherein the controller comprises a microcontroller including an analog frontend for ECG acquisition, a digital signal processor configured to sample at least 100 samples per second with 10-bit resolution, and a wireless communication module configured to communicate with a smartphone application or computer GUI.

6. The system of claim 1, wherein the controller executes a threshold algorithm that blocks stimulation if noise dominates the ECG, if arrhythmia is detected, or if the ECG electrodes are not connected to the patient.

7. The system of claim 1, wherein the controller regulates current to the at least one electromagnet and the at least one light-emitting diode such that step variations are limited and a stimulation profile is generated that reflects the ECG signal.

8. The system of claim 1, wherein the stimulator array is configured to provide amplitude-modulated stimulation that mirrors the ECG signal and frequency-modulated stimulation in which frequency depends on phases of the cardiac cycle, including operation with fixed polarity and with alternating polarity about a zero-field baseline.

9. The system of claim 1, wherein the controller calculates the force momentum of the heart, value β, based on ECG-derived amplitudes of QR, RS, and ST waves and a corrected time tQTc derived from tQT and tRR, and uses the value β to dictate the electromagnetic and optical stimulation.

10. The system of claim 1, wherein the controller is configured to detect electrode contact by injecting a small oscillating signal between electrodes and, upon detecting loss of contact, to pause calculations and stimulations and enter a low-power sleep mode with a visual and/or auditory alert, and further to store ECG data and transmit alerts for detectable anomalies including tachycardia and arrhythmia.

11. A method of determining and modifying the electromagnetic activity of a patient's heart using a portable wearable device, comprising:
providing a sensor array comprising one or more ECG electrodes configured to detect the electromagnetic activity of the patient's heart in real time;
establishing communication between the sensor array and a controller retained in a portable enclosure;
receiving, at the controller, signals from the one or more ECG electrodes;
translating, at the controller, the received signals into sensor data corresponding to the electromagnetic activity of the patient's heart;
executing software on the controller, the software outputting the sensor data to enable a user to read the electromagnetic activity of the patient's heart;
calculating, at the controller, a force momentum of the heart based on the sensor data;
transmitting, from the controller to a stimulator array comprising at least one electromagnet and at least one light-emitting diode, a control signal based on the calculated force momentum;
emitting, from the stimulator array, both electromagnetic and optical signals to the patient's heart in real-time response to the calculated force momentum;
and modifying in real time the electromagnetic and optical signals based on the electromagnetic activity of the heart.

12. The method of claim 11, wherein calculating the force momentum comprises calculating a value β based on ECG-derived amplitudes of QR, RS, and ST waves and a corrected time tQTc derived from tQT and tRR.

13. The method of claim 11, further comprising determining a baseline in which no cardiac contraction occurs and withholding stimulation during the baseline, and increasing stimulation intensity to correspond to increasing ECG amplitude such that stimulation is in phase with the heart's activity.

14. The method of claim 11, wherein emitting comprises generating an amplitude-modulated stimulation that mirrors the ECG signal and, alternatively, generating a frequency-modulated stimulation in which stimulation frequency depends on phases of the cardiac cycle.

15. The method of claim 14, further comprising alternating magnetic field polarity about a zero-field baseline while limiting absolute field magnitude to regulatory limits.

16. The method of claim 11, further comprising sampling the ECG at a rate of at least 200 samples per second with at least 12-bit resolution, digitally filtering mains noise and low-frequency drifts, and extracting a baseline over at least five seconds.

17. The method of claim 11, further comprising blocking stimulation when noise dominates the ECG, when arrhythmia is detected, or when the ECG electrodes are not connected, and pausing calculations and stimulations until reconnection.

18. The method of claim 17, further comprising detecting electrode contact by injecting a small oscillating signal between electrodes and sensing propagation through the skin, and entering a low-power sleep mode with a visual and/or auditory alert upon loss of contact.

19. The method of claim 11, further comprising regulating current to the at least one electromagnet and the at least one light-emitting diode to avoid significant step variations such that the stimulation profile reflects the ECG signal.

20. The method of claim 11, further comprising storing acquired ECG data on a connected smart device, wirelessly transmitting the data to a practitioner or remote platform, and generating configurable alerts for detectable anomalies including tachycardia and arrhythmia, wherein the portable enclosure is implemented as a wearable belt or chest harness positioning the electrodes on opposing sides of the heart.
